(19) United States
(12) Patent Application Publication
(10) Pub. No.: US 2026/0193690 A1
(43) Pub. Date: Jul. 9, 2026

(54) A METHOD FOR DETERMINATION OF D-2-HYDROXYGLUTARATE CONCENTRATION AND KIT

(71) Applicant: THE SECOND HOSPITAL OF SHANDONG UNIVERSITY, Jinan, Shandong (CN)

(72) Inventors: Wen ZHANG, Jinan (CN); Mu YANG, Jinan (CN); Chao GAO, Jinan (CN); Dongqi TANG, Jinan (CN); Jiang LIU, Jinan (CN)

(73) Assignee: THE SECOND HOSPITAL OF SHANDONG UNIVERSITY, Jinan, Shandong (CN)

(21) Appl. No.: 19/128,225

(22) PCT Filed: Apr. 25, 2024
(86) PCT No.: PCT/CN2024/089821

§ 371 Date: May 8, 2025

(30) Foreign Application Priority Data
Jul. 31, 2023 (CN) 202310948779.9

(51) Int. Cl. C12Q 1/32

(52) U.S. Cl. CPC C12Q 1/32

(57) ABSTRACT

A method for determination of D-2-hydroxyglutarate concentration and a kit. The determination method is characterized by taking FAD-dependent D-2-hydroxyglutarate dehydrogenase, resazurin and PBS buffer solution system as the working solution to realize the determination for D-2-hydroxyglutarate in the solid tissue sections, and adding different concentrations of D-2-hydroxyglutarate into the agarose water solution to prepare standard substance sections, while making the standard curve. The concentration of D-2-hydroxyglutarate in the tissue sample is detected after simple treatment for the sample, and the detection results are consistent with the results of mass spectrometry, with high accuracy; it takes 20 minutes to determine the standard substance and prepare the sample section; the detection time of the sample section is about 3 minutes; it takes about 2 minutes to calculate the D-2-HG concentration, and it takes less than 5 minutes after preparing the standard substance curve and sample section.

### FIELD

The present invention relates to the technical field of enzymatic determination, in particular to a method for determination of D-2-hydroxyglutarate concentration and a kit.

### BACKGROUND

Mutations in isocitrate dehydrogenase 1 (IDH1) or paralogue IDH2 (collectively known as IDH) thereof are prevalent in various types of cancer, including low-grade glioma and secondary glioblastoma, acute myeloid leukemia, cholangiocarcinoma, chondrosarcoma, sinonasal undifferentiated carcinoma, angioimmunoblastic T-cell lymphoma, etc. About 80% of low-grade gliomas carry isocitrate dehydrogenase-1 mutations. Mutations in the active site of IDH1 enzyme reduce isocitrate activity significantly, resulting in the production of a new allosteric enzyme, which can catalyze the reduction of a-ketoglutarate (a-KG) to D-2-hydroxyglutarate (also abbreviated as D-2-HG), so as to accumulate D-2-hydroxyglutarate in tumors, even if the content of D-2-hydroxyglutarate in IDH-mutated tumors is 50~100 times higher than that in normal tissues.

In addition, the concentration of D-2-hydroxyglutarate is also related to non-tumor diseases, such as the presence of an enzyme, D-2-hydroxyglutarate dehydrogenase (D-2-HGDH) in human mitochondria, which uses FAD as a coenzyme to catalyze the oxidative dehydrogenation of D-2-hydroxyglutarate into a-KG, so as to maintain the content of 2-HG in cells at a low level. In case of mutations in D-2-hydroxyglutarate dehydrogenase, D-2-hydroxyglutarate cannot be dehydrogenated to become α-KG, resulting in the accumulation of D-2-hydroxyglutarate. In patients with D-2-hydroxyglutarate dehydrogenase mutations, high accumulation of 2-HG will lead to 2-hydroxyglutaric aciduria (2-HGA). For another example, a study published in Science in 2022 showed that the increase of

D-2-hydroxyglutarate around IDH mutant brain tumors reduced lymphocyte infiltration, and D-2-hydroxyglutarate affected the proliferation and metabolism of CD8+ T cells in the tumor microenvironment to inhibit the immune system.

In many diseases, D-2-hydroxyglutarate or L-2-HG is an abnormal substance in metabolism, which exists in urine, tissue fluid and tissue, and will accumulate abnormally in the body. However, in practice, the results of D-2-hydroxyglutarate content alone cannot correspond to the types of disease one by one, and other detections are needed to detect the types of disease finally.

In addition, D-2-hydroxyglutarate or L-2-hydroxyglutarate concentration can be respectively detected rapidly and accurately, which is of great significance for exploring the pathogenic mechanism, screening suitable drugs to reduce the production of D-2-hydroxyglutarate or L-2-hydroxyglutarate, and exploring the role of D-2-HG or L-2-HG metabolism in protein or polysaccharide synthesis. For example, the invention patent CN201380011978.1 discloses a tool and method for the determination of D-2-hydroxyglutarate or (D) 2-hydroxyhexanedioic acid, and a kit is prepared. In the article named A High-Throughput Fluorimetric Assay for 2-Hydroxyglutarate Identifi es Zaprinast as a Glutaminase Inhibitor, a simple and low-cost fluorescence microplate method is developed based on phosphoglycerate dehydrogenase (PHGDH) to detect D-2-hydroxyglutarate, and the compounds that can inhibit the production of D-2-hydroxyglutarate are screened. In the article named A D-2-hydroxyglutarate biosensor based on specific transcriptional regulator DhdR, a biosensor for the determination of D-2-hydroxyglutarate is developed, which is also used for detecting the concentration of D-2-hydroxyglutarate in bacteria, demonstrating the important role of D-2-hydroxyglutarate metabolism in lipopolysaccharide synthesis of Pseudomonas aeruginosa.

At present, liquid chromatography-mass spectrometry (LC-MS) or gas chromatography-mass spectrometry (GC-MS) is generally used for achieving the concentration measurement of D-2-hydroxyglutarate and L-2-hydroxyglutarate. However, on the one hand, the above-mentioned method requires pre-treatment of samples, which is tedious and time-consuming; on the other hand, the mass spectrometry machine is expensive, which greatly increases the detection cost.

The invention patent CN112592958B discloses a method for determination of D-2-hydroxyglutarate by using FAD-dependent D-2-hydroxyglutarate dehydrogenase and resazurin. During the detection, a new FAD-dependent D-2-hydroxyglutarate dehydrogenase is designed to avoid from adding cofactors, coenzymes or cardioflavase. Compared with conventional detection, the sensitivity of the detection is improved greatly. However, this determination method is more suitable for the determination of liquid samples, such as serum, urine, cerebrospinal fluid, etc., which can be detected in the fluorescent enzyme labeled instrument only after protein removal. However, in case of detection for the tissue of solid sample, pre-treatment shall be performed for the solid sample, e.g. the solid sample is crushed, D-2-hydroxyglutarate is released from the solid sample for detection, so the detection accuracy and efficiency will be affected by the pre-treatment.

Therefore, a rapid, accurate and efficient method for determination of D-2-hydroxyglutarate concentration in the solid samples is urgently needed.

### SUMMARY

In order to solve the above problems, the purpose of the present invention is to provide a method for determination of D-2-hydroxyglutarate concentration and a kit.

The purpose of the present invention is realized by the following technical solution:

The coding gene sequence, amino acid sequence and preparation method of FAD-dependent D-2-hydroxyglutarate dehydrogenase (FAD-dependent D-2-HGDH) adopted in the present invention have been disclosed in CN112592958B and are not described in this patent.

A method for determination of D-2-hydroxyglutarate concentration, characterized by taking FAD-dependent D-2-hydroxyglutarate dehydrogenase, resazurin and PBS buffer solution system as the working solution to realize the determination for D-2-hydroxyglutarate in the solid tissue sections; The determination method of the present invention is for the purpose of non-disease diagnosis and treatment.

A method for determination of D-2-hydroxyglutarate concentration, comprising the following steps:

\textcircled{1} preparing standard substance sections of D-2-hydroxyglutarate, adding the D-2-hydroxyglutarate into agarose water solution, heating and stirring till reaching the clear and transparent state, pouring the solution onto a gel plate to solidify and take out, cutting it into a square with a cross-sectional area of 0.1~1.5 cm×0.1~1.5 cm, and placing them in a tissue tray of a freezing microtome, and covering an agarose gel block with an OCT freezing medium; after the OCT freezing medium is frozen as solid in the freezing microtome, cutting the agarose gel to reach a thickness of 5~25 μm, placing them on an anti-slip glass slide, cutting them into cuboids with a cross-sectional area of 0.1~1 cm×0.1~1 cm, and storing them in a refrigerator at −20° C. to obtain a series of standard substance sections of D-2-hydroxyglutarate with different concentrations for standby;
wherein the ratio of D-2-hydroxyglutarate to agarose water solution is 0~5.0 μmol: 1 g;
three groups of parallel experiments are set for each concentration of standard substance sections;
\textcircled{2} slicing of tissue samples to be detected taking out the tissue to be detected from the refrigerator at −80° C., placing it onto the ice, and then placing it in the tissue tray of the freezing microtome, adding the OCT freezing medium to the tissue to be detected; after the OCT freezing medium turns white, slicing the tissue to be detected to a thickness of 5~25 μm, placing it on the anti-slip glass slide, and cutting it into cuboids with a cross-sectional area of 0.1~1 cm×0.1~1 cm, so as to obtain the tissue sections to be detected for standby;
three groups of parallel experiments are set for each tissue sample section to be detected;
\textcircled{3} preparation of staining solution: the staining solution is a buffer solution with the pH of 7.0~8.0, containing resazurin and FAD-dependent D-2-hydroxyglutarate dehydrogenase; the buffer solution can be Tris-Hel buffer solution, PBS buffer solution or HEPES buffer solution; further preferably, it is 1/15 mol/L of PBS buffer solution; the concentration range of the resazurin is 0.5 μM-200 μM, and the concentration range of the FAD-dependent D-2-hydroxyglutarate dehydrogenase is 1.3 μg/mL~520 μg/ml;
preferably, the staining solution is a PBS buffer solution (with a mass concentration of 1/15 mol/L) with pH=7.4, which contains 10UM of resazurin and 26 ng/ml of FAD-dependent D-2-hydroxyglutarate dehydrogenase;
\textcircled{4} staining of tissue sections

taking 15 μl~100 μl of the staining solution obtained from Step \textcircled{3}, covering it completely on the tissue sections to be detected and obtained from Step \textcircled{2}, detecting the fluorescence value of the staining solution on the glass slide with a fluorescence detector for 1~10 minutes;
\textcircled{5} data test and result processing of standard substance

placing the standard substance sections of a series of D-2-hydroxyglutarate with different concentrations in Step \textcircled{1} on the glass slide, taking 15 μl~100 μl of the staining solution obtained in Step \textcircled{3} for covering completely, detecting with the fluorescence detector immediately, wherein the fluorescence detector is characterized by the excitation wavelength of 540 nm and the emission wavelength of 590 nm; taking the value when the fluorescence detector detects for 1~10 minutes as the fluorescence value of the staining solution on the glass slide; drawing the scatter diagram by taking the average fluorescence value of each concentration detected by the fluorescence detector as Value y, and the concentration of D-2-hydroxyglutarate as Value x, and forming the standard substance curve y=ax+b according to the scatter diagram;

and calculating the content of D-2-hydroxyglutarate in the tissue sections to be detected according to the fluorescence value obtained in Step \textcircled{3}.

Preferably, preparing standard substance sections of D-2-hydroxyglutarate, preparing D-2-hydroxyglutarate into a series of agarose water solution with a concentration range of 0~2.0 μmol/g, heating the agarose water solution till reaching the clear and transparent state, pouring the solution onto a gel plate to solidify and take out, cutting it into cuboids with a cross-sectional area of 0.1~1.5 cm×0.1~1.5 cm, and placing them in a tissue tray of a freezing microtome, and covering an agarose gel block with an OCT freezing medium (the appropriate amount of OCT freezing medium is to uniformly cover the agarose gel block, and the amount is generally 350~450 μL); after the OCT freezing medium is frozen as solid in the freezing microtome, cutting the agarose gel to reach a thickness of 5~25 μm, placing them on the slides, cutting them into a square with a cross-sectional area of 0.1~1 cm×0.1~1 cm, and storing them in a refrigerator at −20° C. to obtain a series of standard substance sections of D-2-hydroxyglutarate with different concentrations for standby;

in the process of cutting it into cuboids with a cross-sectional area of 0.1~1 cm×0.1~1 cm, it can be cut into a large cuboid first, and then the part with the edge in contact with the OCT freezing medium can be cut, so as to avoid the influence of the OCT freezing medium on the detection results of the agarose gel block.

Three groups of parallel experiments are set for each concentration of standard substance sections;
preferably, the concentration of D-2-hydroxyglutarate in the standard substance section is a gradient concentration in the range of 0~5.0 μmol/g.
further preferably, the concentrations of D-2-hydroxyglutarate in the standard substance section are 0 μmol/g, 0.1 μmol/g, 0.25 μmol/g, 0.5 μmol/g, 1.0 μmol/g, 1.5 μmol/g and 2.0 μmol/g.
preferably, after the staining solution is added to the standard substance section and the tissue section to be detected, the detection time of the fluorescence detector is the same, 1-3 minutes;
further preferably, the detection time of the fluorescence detector is 3 minutes.

The present invention also comprises a kit obtained by using the method for determination of D-2-hydroxyglutarate concentration, containing buffer solution with pH7.0~8.0, mother liquor of resazurin, mother liquor of FAD-dependent D-2-hydroxyglutarate dehydrogenase, and agarose gel slides containing the serial concentration of D-2-hydroxyglutarate; the concentration range of the mother liquor in the kit is 0.2 mM~25 mM, the concentration range of the mother liquor of FAD-dependent D-2-hydroxyglutarate dehydrogenase in the kit is 1.3 mg/ml-26 mg/ml;
and the buffer solution is Tris-Hcl buffer solution, PBS buffer solution or HEPES buffer solution.

Preferably, the agarose gel containing the serial concentration of D-2-hydroxyglutarate is placed on the anti-slip glass slide, and the concentration range of D-2-hydroxyglutarate in the agarose gel is 0~5.0 μmol/g.

Further preferably, the concentrations of D-2-hydroxyglutarate in the agarose gel are 0 μmol/g, 0.1 μmol/g, 0.25 μmol/g, 0.5 μmol/g, 1.0 μmol/g, 1.5 μmol/g and 2.0 μmol/g.

Preferably, the size and thickness of agarose gel containing the serial concentration of D-2-hydroxyglutarate are cut as required. The agarose gel is rectangle, with the thickness of 5~25 μm and the cross-sectional area of 0.1~1 cm×0.1~1 cm, and the size and thickness of the agarose gel can also be adjusted according to the size of the tissue to be detected.

PBS concentration and pH, resazurin concentration, and enzyme concentration are not the only constant concentrations for work. It is to use the optimization conditions of the aforementioned patent. These conditions cannot become the limitation that other concentrations do not work.

The present invention also comprises a method for using the kit obtained by using the method for determination of D-2-hydroxyglutarate concentration, comprising the following steps:
A. Preparation of staining solution: adding resazurin solution and FAD-dependent D-2-hydroxyglutarate dehydrogenase solution to the PBS buffer solution to prepare the staining solution;
The concentration range of the resazurin mother liquor in the kit is 0.2 mM~25 mM, and the concentration range of the FAD-dependent D-2-hydroxyglutarate dehydrogenase mother liquor is 1.3 mg/ml-26 mg/ml. During the use, the two solutions need to be added to the PBS buffer solution to prepare the staining solution;
The final prepared staining solution is the buffer solution with pH=7.0~8.0, and the staining solution contains 10 μM of resazurin and 26 μg/ml of FAD-dependent D-2-hydroxyglutarate dehydrogenase;
B. Making of standard curve: covering the staining solution obtained in Step A completely onto an agarose gel glass slide containing the serial concentration of D-2-hydroxyglutarate, detecting the fluorescence value of the staining solution on the glass slide with a fluorescence detector immediately, taking the value when the fluorescence detector detects for 1~10 minutes, drawing the scatter diagram by taking the average fluorescence value of each concentration detected by the fluorescence detector as Value y, and the concentration of D-2-hydroxyglutarate as Value x, and drawing the standard curve y=ax+b according to the scatter diagram;
In the process of making the standard curve, the fluorescence detector time for the agarose gel glass slide with each concentration of D-2-hydroxyglutarate shall be the same as far as possible, preferably 1~3 minutes; the excitation wavelength is 540 nm, and the emission wavelength is 590 nm;
C. Taking out the tissue to be detected from the refrigerator at −80° C., placing it onto the ice, and then placing it in the tissue tray of the freezing microtome, adding the OCT freezing medium to the tissue to be detected; after the OCT freezing medium turns white, slicing the tissue to be detected to the same thickness as the agarose gel glass slide, placing it on the slides, and cutting it into a square with the same cross-sectional area as the agarose gel glass slide, so as to obtain the tissue sections to be detected for standby;
three groups of parallel experiments are set for each tissue sample section to be detected;
covering the staining solution obtained from Step A completely onto the tissue sections to be detected, detecting the fluorescence value of the staining solution on the glass slide with the fluorescence detector for 1~10 minutes; preferably, the detection time is the same as that of the fluorescent detector for making the standard curve, and the detection environment and characteristics are the same as the detection time of the fluorescent detector for making the standard curve;
D. The test fluorescence value in Step C is brought into the standard curve in Step B to calculate the content of D-2-hydroxyglutarate in the tissue to be detected.

Compared with the prior art, the present invention has the advantages as follows:

The method for determination of D-2-hydroxyglutarate concentration in the present invention is characterized into that the concentration of D-2-hydroxyglutarate in the tissue samples can be detected after simple treatment, and the detection results are consistent with the results of the “golden standard” detection method (mass spectrometry) with high accuracy. It takes 5~6 hours from sample treatment to calculation of D-2-hydroxyglutarate concentration by the conventional mass spectrometry. However, the method for determination of D-2-hydroxyglutarate concentration in the present invention can prepare the standard substance in advance and store it in the refrigerator at −20° C., it takes 20 minutes to detect the standard substance and prepare the sample section, the sample section detection time is about 3 minutes, and it takes about 2 minutes to calculate the D-2-HG concentration, and it only takes less than 5 minutes after preparing the standard substance curve and sample section, which greatly shortens the detection time and reduce the detection cost.

The kit obtained by using the method for determination of D-2-hydroxyglutarate concentration in the present invention contains PBS buffer solution, resazurin solution, FAD-dependent D-2-hydroxyglutarate dehydrogenase solution, and agarose gel slides containing the serial concentration of D-2-hydroxyglutarate; the standard curve can be prepared, and the user only needs to prepare the tissue to be detected, which is convenient for detection, greatly shortens the detection time, reduces the detection cost, and is convenient for promotion and use.

### DESCRIPTION OF THE DRAWINGS

FIG. 1 shows a schematic diagram of influence on enzyme activity reaction by taking OCT freezing medium as the medium;

FIG. 2 shows a schematic diagram of influence on enzyme activity reaction by taking agarose as the medium;

FIG. 3 shows a schematic diagram of influence on enzyme activity reaction by taking OCT freezing medium as the medium;

FIG. 4 shows a schematic diagram of influence of different buffer solutions on enzyme reaction activity;

FIG. 5 shows a schematic diagram of influence of different solution areas on standard curve;

FIG. 6 shows a schematic diagram of influence of different solution thicknesses on standard curve;

FIG. 7 shows a standard substance range while detecting the standard substance at different time nodes;

FIG. 8 shows a schematic diagram of standard curve in Embodiment 1; and

FIG. 9 shows a comparison diagram of data obtained by using the determination method in the present invention and the traditional mass spectrometry determination method.

### DETAILED DESCRIPTION OF THE EMBODIMENTS

The purpose of the present invention is to provide a method for determination of D-2-hydroxyglutarate concentration and a kit, and realized by the following technical solution:

The resazurin used in the embodiment of the present invention is a product purchased from sigma brand.

At the beginning of the design of the method for determination of D-2-hydroxyglutarate concentration, the medium of D-2-HG standard substance is selected, and the types of media available for selection include OCT freezing medium, agarose and polyacrylamide (PAGE) solution, and the process is as follows:
I. Influence of different D-2-HG standard media on reaction rate
(1) Preparation of D-2-HG standard media
i. OCT freezing medium: prepare 1/15 M of PBS solution with the mass concentration of 0% OCT, 0.1% OCT, 0.2% OCT, 0.5% OCT and 5% OCT.
ii. Agarose: prepare 1/15M of PBS solution with the agarose mass concentration of 0%, 0.05%, 0.1% and 0.2%;
iii. PAGE solution: prepare 1/15M of PBS solution with the mass concentration of 0% PAGE, 0.1% PAGE, 1% PAGE and 5% PAGE;
(2) Enzyme reaction system

| | |
|---|---|
| 1/15M PBS (or the above media that | 183 μl |
| has been configured) | |
| FAD-dependent D-2-hydroxyglutarate | 5 μl |
| dehydrogenase (32.5 μg/ml) | |
| 1 mM resazurin | 10 μl |
| 1 mM D-2-HG | 2 μl |
| Total | 200 μl |

(3) After the above-mentioned solution is prepared, it is placed on the 96-well blackboard, and the emission light of 540 nm/excitation light of 590 nm is detected in the Biotek Cytation5 instrument. The detection is set to be performed every 2s for a total of 300s.

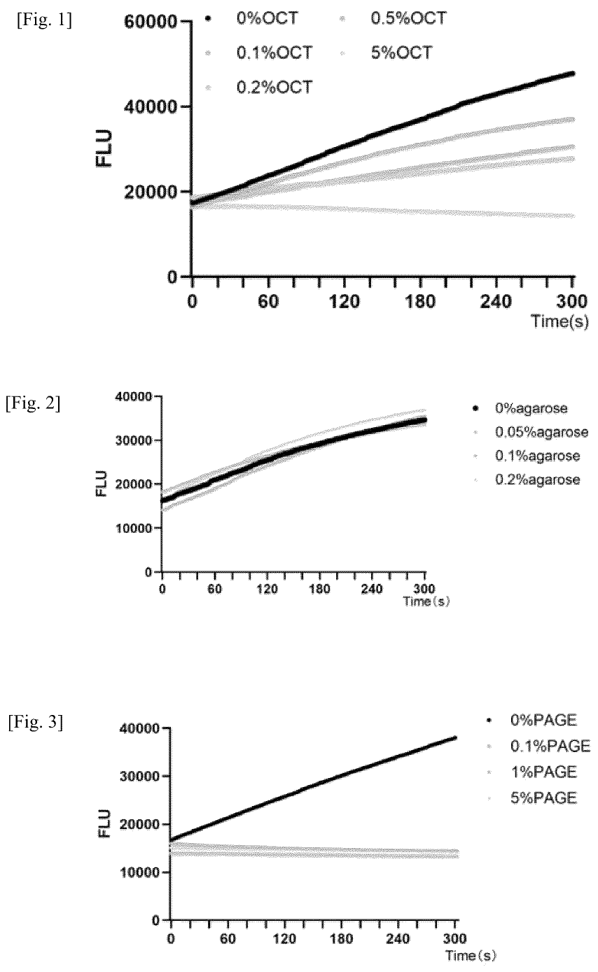

The results are shown in FIG. 1~FIG. 3. The results in FIG. 1 indicate that OCT freezing medium has obvious inhibition on enzyme activity reaction, and is not suitable as a medium for D-2-HG standard substance; the results in FIG. 2 show that agarose has no inhibitory effect on the enzymatic reaction and can be used as a medium for D-2-hydroxyglutarate standard substance; and the results of FIG. 3 show that the components in PAGE gel can significantly inhibit the enzyme activity reaction and are not suitable as a medium for D-2-hydroxyglutarate standard substance. Therefore, the agarose gel is selected as the medium for D-2-hydroxyglutarate standard substance.

In the present invention, some experimental items and conditions are optimized and tested as follows:
II. Test for Different Buffer Solutions in Staining Solution
(1) Enzyme reaction system

| | |
|---|---|
| Buffer solution | 183 μl |
| FAD-dependent D-2-hydroxyglutarate | 5 μl |
| dehydrogenase (32.5 μg/ml) | |
| 1 mM/2 mM/10 mM/25 mM/50 mM/100 mM resazurin | 10 μl |
| 1 mM D-2-HG | 2 μl |
| Total | 200 μl |

The above-mentioned buffer solution is 1/15M of PBS solution, 0.1 M of Tris-HCl buffer solution or 0.1M of HEPES buffer solution;
(2) After the above-mentioned solution is prepared, it is placed on the 96-well blackboard, and the emission light of 540 nm/excitation light of 590 nm is detected in the Biotek Cytation5 instrument. The detection is set to be performed every 2s for a total of 300s.

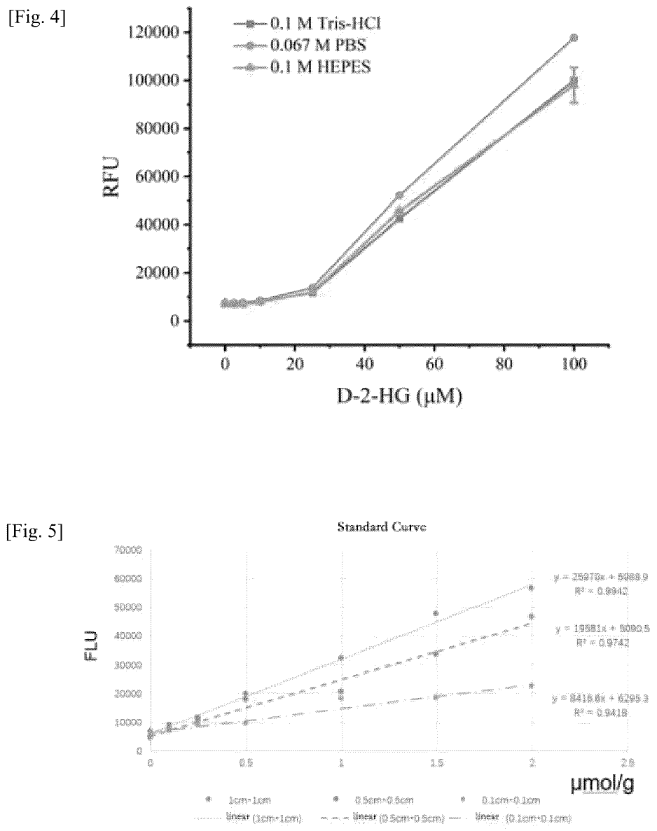

(3) The results are shown in FIG. 4, all the 0.1M Tris-HCl buffer solution, 0.1M HEPES buffer solution and 1/15M PBS buffer solution can be increased with the increase of D-2-hydroxyglutarate concentration, wherein 1/15 M PBS buffer solution has higher fluorescence value and better performance at the same concentration of resazurin. Therefore, 1/15 M PBS buffer solution is preferred for subsequent experiments.
III. Test for Section Area of Different D-2-hydroxyglutarate (D-2-HG) standard substances and Tissue Samples in Frozen Sections
1. Fluorescence detector

The fluorescence detector adopts the instrument (BLP-FD-100CS) customized by wave-particle photon, which integrates excitation light, beam collimation, uniform light, extinction, fluorescence collection, fluorescence detection, filtering and signal shielding protection into one unit, with the excitation wavelength of 540 nm, the emission wavelength of 590 nm, the optical density of OD5, the spot diameter of 6.5 mm, the input voltage of 5V, the analog signal output of 0-3.3V, and digital signal output.
2. D-2-HG standard substance treatment:
(1) Production of D-2-HG standard substance: prepare 3% agarose solution containing D-2-HG 0 μmol/g, 0.1 μmol/g, 0.25 μmol/g, 0.5 μmol/g, 1 μmol/g, 1.5 μmol/g and 2.0 μmol/g (20 ml for each) (in order to convert g/cm³, the density of brain tumor tissue refers to 1.054 g/cm³ of normal brain tissue). Use the microwave oven for heating, and heat 3% agarose solution containing D-2-HG at each concentration at the medium baking temperature till it is clear and transparent, then pour it into the gel plate to solidify and take out after half an hour, and cut the solidified agarose into cuboids with a cross-sectional area of 1.5 cm*1.5 cm. Place them in the tissue tray of the freezing microtome (Leica), and cover the agarose gel block with 400 μl of OCT freezing medium. After the OCT freezing medium is frozen as solid in the freezing microtome, cut the agarose gel to reach a thickness of 20 μm, place them on an anti-slip glass slide, divide them into three groups of tissue sections with a size of 0.1 cm*0.1 cm, 0.5 cm*0.5 cm and 1 cm*1 cm at each agarose concentration. During the slicing, slice the tissue into pieces with a large area first, and then trim them to a size of 0.1 cm*0.1 cm, 0.5 cm*0.5 cm and 1 cm*1 cm. In this way, the tissue sections contaminated with OCT can be removed and the influence of OCT freezing medium on the detection data can be avoided. After the operation is complete, store the cut tissue sections in the refrigerator at −20° C.
(2) Staining of D-2-HG standard substance sections
i. Preparation of staining solution: 500 μl 1/15M PBS+1 μl 5 mM resazurin+1 μl 13 mg/ml FAD-dependent D-2-hydroxyglutarate dehydrogenase;
ii. Add 50 μl of staining solution onto the tissue at the room temperature of 25° C. in the dark environment to cover the D-2-HG standard substance completely, and use the Fluorescence detector to detect the fluorescence of the staining solution on the glass slide every 2 s for a total of 300 s.
3. Making of standard curve

Draw the scatter diagram by taking the D-2-HG concentration of 0 μmol/g, 0.1 μmol/g, 0.25 μmol/g, 0.5 μmol/g, 1 μmol/g, 1.5 μmol/g, 2.0 μmol/g of D-2-HG in the standard substance sections with the section area of 0.1 cm*0.1 cm as Value x, and the fluorescence value detected by the above method at the time point of 180 s as Value y, and form the fitting linear relationship of y=ax+b according to the scatter diagram, as shown in FIG. 4.

Draw the scatter diagram by taking the D-2-HG concentration of 0 μmol/g, 0.1 μmol/g, 0.25 μmol/g, 0.5 μmol/g, 1 μmol/g, 1.5 μmol/g, 2.0 μmol/g of D-2-HG in the standard substance sections with the section area of 0.5 cm*0.5 cm as Value x, and the fluorescence value detected by the above method at the time point of 180s as Value y, and form the fitting linear relationship of y=ax+b according to the scatter diagram, as shown in FIG. 4.

Draw the scatter diagram by taking the D-2-HG concentration of 0 μmol/g, 0.1 μmol/g, 0.25 μmol/g, 0.5 μmol/g, 1 μmol/g, 1.5 μmol/g, 2.0 μmol/g of D-2-HG in the standard substance sections with the section area of 1 cm*1 cm as Value x, and the fluorescence value detected by the above method at the time point of 180s as Value y, and form the fitting linear relationship of y=ax+b according to the scatter diagram, as shown in FIG. 5. The results show that when the section area is 0.1 cm*0.1 cm, 0.5 cm*0.5 cm and 1 cm*1 cm, the fluorescence value is positively correlated with D-2-HG, which can be used as the cross-sectional area of the standard substance section.
III. Test for Section Thickness of Different D-2-HG standard substances and Tissue Samples in Frozen Sections
1. Fluorescence detector

The fluorescence detector adopts the instrument (BLP-FD-100CS) customized by wave-particle photon, with the parameters the same as above.
2. D-2-HG standard substance treatment:
(1) Production of D-2-HG standard substance: prepare 3% agarose solution containing D-2-HG 0 μmol/g, 0.1 μmol/g, 0.25 μmol/g, 0.5 μmol/g, 1 μmol/g, 1.5 μmol/g, 2.0 μmol/g (20 ml for each) (in order to convert g/cm³, the density of brain tumor tissue refers to 1.054 g/cm³ of normal brain tissue). Use the microwave oven for heating, and heat 3% agarose solution containing D-2-HG at each concentration at the medium baking temperature till it is clear and transparent, then pour it into the gel plate to solidify and take out after half an hour, and cut the solidified agarose into cuboids with a cross-sectional area of 1.5 cm*1.5 cm. Place them in the tissue tray of the freezing microtome (Leica), and cover the agarose gel block with a small amount of OCT freezing medium. After the OCT freezing medium is frozen as solid in the freezing microtome, cut the agarose gel to reach three thickness gradients of 5 μm, 20 μm and 25 μm, place them on the anti-slip glass slide, divide them into cuboids with a cross-sectional area of 0.5 cm*0.5 cm, and store them in a refrigerator at −20° C.
(2) Staining of D-2-HG standard substance sections
i. Preparation of staining solution: 500 μl 1/15M PBS+1 μl 5 mM resazurin+1 μl 13 mg/ml FAD-dependent D-2-hydroxyglutarate dehydrogenase;
ii. Add 50 μl of staining solution onto the tissue at the room temperature of 25° C. in the dark environment to cover the D-2-HG standard substance completely, and use the Fluorescence detector to detect the fluorescence of the staining solution on the glass slide every 2s for a total of 300s.
3. Making of standard curve

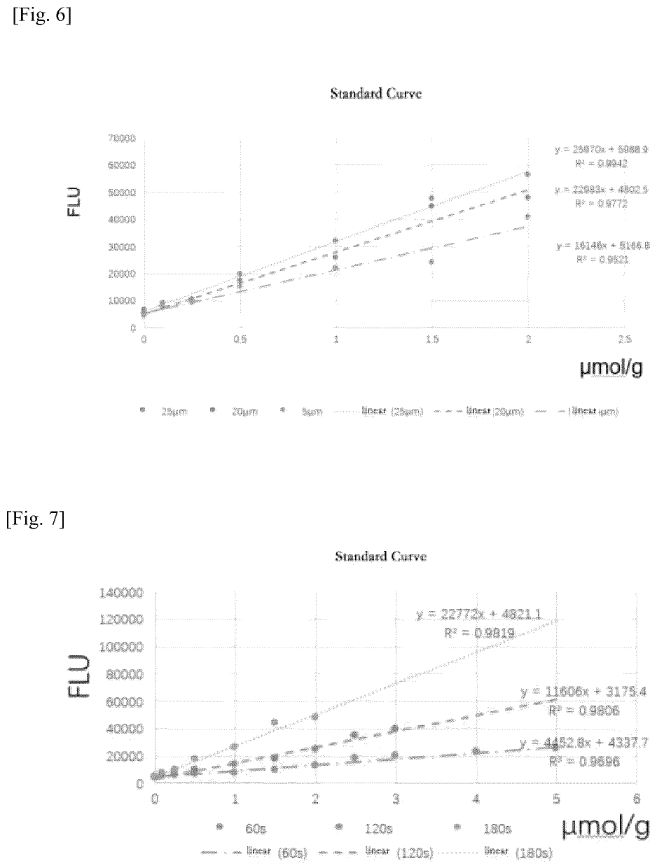

Draw the scatter diagram by taking the D-2-HG concentration of 0 μmol/g, 0.1 μmol/g, 0.25 μmol/g, 0.5 μmol/g, 1 μmol/g, 1.5 μmol/g, 2.0 μmol/g of D-2-HG in the standard substance sections with the section thickness of 5 μm as Value x, and the fluorescence value detected by the above method at the time point of 180s as Value y, and form the fitting linear relationship of y=ax+b according to the scatter diagram, and the results are as shown in FIG. 6.

Draw the scatter diagram by taking the D-2-HG concentration of 0 μmol/g, 0.1 μmol/g, 0.25 μmol/g, 0.5 μmol/g, 1 μmol/g, 1.5 μmol/g, 2.0 μmol/g of D-2-HG in the standard substance sections with the section thickness of 20 μm as Value x, and the fluorescence value detected by the above method at the time point of 180s as Value y, and form the fitting linear relationship of y=ax+b according to the scatter diagram, and the results are as shown in FIG. 6.

Draw the scatter diagram by taking the D-2-HG concentration of 0 μmol/g, 0.1 μmol/g, 0.25 μmol/g, 0.5 μmol/g, 1 μmol/g, 1.5 μmol/g, 2.0 μmol/g of D-2-HG in the standard substance sections with the section thickness of 25 μm as Value x, and the fluorescence value detected by the above method at the time point of 180s as Value y, and form the fitting linear relationship of y=ax+b according to the scatter diagram, and the results are as shown in FIG. 6. It can be seen that when the section thickness is 5 μm, 20 μm and 25 μm, the fluorescence value is positively correlated with D-2-HG, which can be used as the section thickness of the standard substance section.
IV. Test for Different Concentrations of Resazurin in the Staining Solution
1. Fluorescence detector

The fluorescence detector adopts the instrument (BLP-FD-100CS) customized by wave-particle photon, which integrates excitation light, beam collimation, uniform light, extinction, fluorescence collection, fluorescence detection, filtering and signal shielding protection into one unit, with the excitation wavelength of 540 nm, the emission wavelength of 590 nm, the optical density of OD5, the spot diameter of 6.5 mm, the input voltage of 5V, the analog signal output of 0-3.3V, and digital signal output.
2. D-2-HG standard substance treatment:
(1) Production of D-2-HG standard substance: prepare 3% agarose solution containing D-2-HG 0 μmol/g, 0.1 μmol/g, 0.25 μmol/g, 0.5 μmol/g, 1 μmol/g, 1.5 μmol/g and 2.0 μmol/g (20 ml for each) (in order to convert g/cm³, the density of brain tumor tissue refers to 1.054 g/cm³ of normal brain tissue). Use the microwave oven for heating, and heat 3% agarose solution containing D-2-HG at each concentration at the medium baking temperature till it is clear and transparent, then pour it into the gel plate to solidify and take out after half an hour, and cut the solidified agarose into cuboids with a cross-sectional area of 1.5 cm*1.5 cm. Place them in the tissue tray of the freezing microtome (Leica), and cover the agarose gel block with 400 μl of OCT freezing medium. After the OCT freezing medium is frozen as solid in the freezing microtome, cut the agarose gel to reach a thickness of 20 μm, place them on an anti-slip glass slide, divide them into three groups of tissue sections with a size of 0.5 cm*0.5 cm at each agarose concentration. During the slicing, slice the tissue into pieces with a large area first, and then trim them to a size of 0.5 cm*0.5 cm. In this way, the tissue sections contaminated with OCT can be removed and the influence of OCT freezing medium on the detection data can be avoided. After the operation is complete, store the cut tissue sections in the refrigerator at −20° C.
(2) Staining of D-2-HG standard substance sections
i. The staining solution is divided into five groups as below:
\textcircled{1} 498.5 μl 1/15M PBS+0.5 μl 0.5 mM resazurin+1 μl 13 mg/ml FAD-dependent D-2-hydroxyglutarate dehydrogenase
\textcircled{2} 498.5 μl 1/15M PBS+0.5 μl 5 mM resazurin+1 μl 13 mg/ml FAD-dependent D-2-hydroxyglutarate dehydrogenase
\textcircled{3} 498.5 μl 1/15M PBS+1 μl 5 mM resazurin+1 μl 13 mg/ml FAD-dependent D-2-hydroxyglutarate dehydrogenase
\textcircled{4} 489.5 μl 1/15M PBS+10 μl 5 mM resazurin+1 μl 13 mg/ml FAD-dependent D-2-hydroxyglutarate dehydrogenase
\textcircled{5} 479.5 μl 1/15M PBS+20 μl 5 mM resazurin+1 μl 13 mg/ml FAD-dependent D-2-hydroxyglutarate dehydrogenase
i. Add 50 μl of staining solution onto the tissue at the room temperature of 25° C. in the dark environment to cover the D-2-HG standard substance completely, and use the Fluorescence detector to detect the fluorescence of the staining solution on the glass slide every 2s for a total of 300s.
3. Result handling

For the above five groups of staining solution, the concentration of resazurin in each group is different, with the concentration range of 0.5 μM-200 μM. The final results show that under the same D-2-HG concentration standard substance, the fluorescence value is as follows: Group \textcircled{3}>Group \textcircled{2}>Group \textcircled{1}, and the fluorescence value of Group \textcircled{4} and Group \textcircled{5} is basically equal to the fluorescence value of Group \textcircled{3}. The results indicate that the concentration of resazurin in Groups \textcircled{1} and \textcircled{2} is insufficient, the concentration of resazurin in Groups \textcircled{4} and \textcircled{5} is too high, and the concentration of resazurin in Group \textcircled{3} is appropriate. The determination of D-2-hydroxyglutarate concentration is feasible in all the five groups, and the optimal ratio belongs to Group \textcircled{3}.
V. Test for Different Concentrations of FAD-dependent D-2-hydroxyglutarate Dehydrogenase in the Staining Solution
1. Fluorescence detector

The fluorescence detector adopts the instrument (BLP-FD-100CS) customized by wave-particle photon, which integrates excitation light, beam collimation, uniform light, extinction, fluorescence collection, fluorescence detection, filtering and signal shielding protection into one unit, with the excitation wavelength of 540 nm, the emission wavelength of 590 nm, the optical density of ODS, the spot diameter of 6.5 mm, the input voltage of 5V, the analog signal output of 0-3.3V, and digital signal output.
2. D-2-HG standard substance treatment:
(1) Production of D-2-HG standard substance: prepare 3% agarose solution containing D-2-HG 0 μmol/g, 0.1 μmol/g, 0.25 μmol/g, 0.5 μmol/g, 1 μmol/g, 1.5 μmol/g and 2.0 μmol/g (20 ml for each) (in order to convert g/cm³, the density of brain tumor tissue refers to 1.054 g/cm³ of normal brain tissue). Use the microwave oven for heating, and heat 3% agarose solution containing D-2-HG at each concentration at the medium baking temperature till it is clear and transparent, then pour it into the gel plate to solidify and take out after half an hour, and cut the solidified agarose into cuboids with a cross-sectional area of 1.5 cm*1.5 cm. Place them in the tissue tray of the freezing microtome (Leica), and cover the agarose gel block with 400 μl of OCT freezing medium. After the OCT freezing medium is frozen as solid in the freezing microtome, cut the agarose gel to reach a thickness of 20 μm, place them on an anti-slip glass slide, divide them into three groups of tissue sections with a size of 0.5 cm*0.5 cm at each agarose concentration. During the slicing, slice the tissue into pieces with a large area first, and then trim them to a size of 0.5 cm*0.5 cm. In this way, the tissue sections contaminated with OCT can be removed and the influence of OCT freezing medium on the detection data can be avoided. After the operation is complete, store the cut tissue sections in the refrigerator at −20° C.
(2) Staining of D-2-HG standard substance sections
i. The staining solution is divided into five groups as below:
\textcircled{1} 498.5.5 μl 1/15M PBS+1 μl 5 mM resazurin+1 μl 1.3 mg/ml FAD-dependent D-2-hydroxyglutarate dehydrogenase
\textcircled{2} 498.5.5 μl 1/15M PBS+1 μl 5 mM resazurin+1 μl 13 mg/ml FAD-dependent D-2-hydroxyglutarate dehydrogenase
\textcircled{3} 498.5 μl 1/15M PBS+1 μl 5 mM resazurin+1 μl 13 mg/ml FAD-dependent D-2-hydroxyglutarate dehydrogenase
\textcircled{4} 489.5 μl 1/15M PBS+1 μl 5 mM resazurin+1 μl 13 mg/ml FAD-dependent D-2-hydroxyglutarate dehydrogenase
\textcircled{5} 479.5 μl 1/15M PBS+1 μl S mM resazurin+1 μl 13 mg/ml FAD-dependent D-2-hydroxyglutarate dehydrogenase
ii. Add 50 μl of staining solution onto the tissue at the room temperature of 25° C. in the dark environment to cover the D-2-HG standard substance completely; 5 min later, use the Fluorescence detector to detect the fluorescence of the staining solution on the glass slide every 2s for a total of 300s.
3. Result handling

For the above five groups of staining solution, the concentration of FAD-dependent D-2-hydroxyglutarate dehydrogenase in each group is different, with the concentration range of 1.3 ug/ml-520 ug/ml. The final results show that under the same D-2-HG concentration standard substance, the fluorescence value is as follows: Group \textcircled{3}>Group \textcircled{2}>Group \textcircled{1}, and the fluorescence value of Group \textcircled{4} and Group \textcircled{5} is basically equal to the fluorescence value of Group 3). The results indicate that the concentration of FAD-dependent D-2-hydroxyglutarate dehydrogenase in Groups \textcircled{1} and \textcircled{2} is insufficient, the concentration of FAD-dependent D-2-hydroxyglutarate dehydrogenase in Groups \textcircled{4} and \textcircled{5} is too high, and the concentration of FAD-dependent D-2-hydroxyglutarate dehydrogenase in Group \textcircled{3} is appropriate. The determination of D-2-hydroxyglutarate concentration is feasible in all the five groups, and the optimal ratio belongs to Group \textcircled{3}.
VI. Test for Different Concentrations of Resazurin in the Kit
1. Fluorescence detector

The fluorescence detector adopts the instrument (BLP-FD-100CS) customized by wave-particle photon, which integrates excitation light, beam collimation, uniform light, extinction, fluorescence collection, fluorescence detection, filtering and signal shielding protection into one unit, with the excitation wavelength of 540 nm, the emission wavelength of 590 nm, the optical density of ODS, the spot diameter of 6.5 mm, the input voltage of 5V, the analog signal output of 0-3.3V, and digital signal output.
2. D-2-HG standard substance treatment:
(1) Production of D-2-HG standard substance: prepare 3% agarose solution containing D-2-HG 0 μmol/g, 0.1 μmol/g, 0.25 μmol/g, 0.5 μmol/g, 1 μmol/g, 1.5 μmol/g and 2.0 μmol/g (20 ml for each) (in order to convert g/cm³, the density of brain tumor tissue refers to 1.054 g/cm³ of normal brain tissue). Use the microwave oven for heating, and heat 3% agarose solution containing D-2-HG at each concentration at the medium baking temperature till it is clear and transparent, then pour it into the gel plate to solidify and take out after half an hour, and cut the solidified agarose into cuboids with a cross-sectional area of 1.5 cm*1.5 cm. Place them in the tissue tray of the freezing microtome (Leica), and cover the agarose gel block with 400 μl of OCT freezing medium. After the OCT freezing medium is frozen as solid in the freezing microtome, cut the agarose gel to reach a thickness of 20 μm, place them on an anti-slip glass slide, divide them into three groups of tissue sections with a size of 0.5 cm*0.5 cm at each agarose concentration. During the slicing, slice the tissue into pieces with a large area first, and then trim them to a size of 0.5 cm*0.5 cm. In this way, the tissue sections contaminated with OCT can be removed and the influence of OCT freezing medium on the detection data can be avoided. After the operation is complete, store the cut tissue sections in the refrigerator at −20° C.
(2) Staining of D-2-HG standard substance sections
i. The staining solution is divided into five groups as below:
\textcircled{1} 474.5 μl 1/15M PBS+25 μl 0.2 mM resazurin+1 μl 13 mg/ml FAD-dependent D-2-hydroxyglutarate dehydrogenase
\textcircled{2} 489.5 μl 1/15M PBS+10 μl 0.5 mM resazurin+1 μl 13 mg/ml FAD-dependent D-2-hydroxyglutarate dehydrogenase
\textcircled{3} 496.5 μl 1/15M PBS+5 μl 1 mM resazurin+1 μl 13 mg/ml FAD-dependent D-2-hydroxyglutarate dehydrogenase
\textcircled{4} 498.5 μl 1/15M PBS+1 μl 5 mM resazurin+1 μl 13 mg/ml FAD-dependent D-2-hydroxyglutarate dehydrogenase
\textcircled{5} 498.8.5 μl 1/15M PBS+0.2 μl 25 mM resazurin+1 μl 13 mg/ml FAD-dependent D-2-hydroxyglutarate dehydrogenase
ii. Add 50 μl of staining solution onto the tissue at the room temperature of 25° C. in the dark environment to cover the D-2-HG standard substance completely, and use the Fluorescence detector to detect the fluorescence of the staining solution on the glass slide every 2s for a total of 300s.
3. Result handling

Draw the scatter diagram by taking the D-2-HG concentration of 0 μmol/g, 0.1 μmol/g, 0.25 μmol/g, 0.5 μmol/g, 1 μmol/g, 1.5 μmol/g, 2.0 μmol/g of D-2-HG by adding 50 μl of the above-mentioned five groups of different staining solution in the standard substance sections with the section thickness of 20 μm as Value x, and the fluorescence value detected by the above method at the time point of 180s as Value y, and form the fitting linear relationship of y=ax+b according to the scatter diagram. There is no significant difference in the standard curves of the five groups of different staining solution (p>0.05). The results indicate that the concentration range of resazurin in the kit is 0.2 mM-25 mM.
VII. Test for Different Concentrations of FAD-dependent D-2-hydroxyglutarate Dehydrogenase in the Kit
1. Fluorescence detector

The fluorescence detector adopts the instrument (BLP-FD-100CS) customized by wave-particle photon, which integrates excitation light, beam collimation, uniform light, extinction, fluorescence collection, fluorescence detection, filtering and signal shielding protection into one unit, with the excitation wavelength of 540 nm, the emission wavelength of 590 nm, the optical density of ODS, the spot diameter of 6.5 mm, the input voltage of SV, the analog signal output of 0-3.3V, and digital signal output.
2. D-2-HG standard substance treatment:
(1) Production of D-2-HG standard substance: prepare 3% agarose solution containing D-2-HG 0 μmol/g, 0.1 μmol/g, 0.25 μmol/g, 0.5 μmol/g, 1 μmol/g, 1.5 μmol/g and 2.0 μmol/g (20 ml for each) (in order to convert g/cm³, the density of brain tumor tissue refers to 1.054 g/cm³ of normal brain tissue). Use the microwave oven for heating, and heat 3% agarose solution containing D-2-HG at each concentration at the medium baking temperature till it is clear and transparent, then pour it into the gel plate to solidify and take out after half an hour, and cut the solidified agarose into cuboids with a cross-sectional area of 1.5 cm*1.5 cm. Place them in the tissue tray of the freezing microtome (Leica), and cover the agarose gel block with 400 μl of OCT freezing medium. After the OCT freezing medium is frozen as solid in the freezing microtome, cut the agarose gel to reach a thickness of 20 μm, place them on an anti-slip glass slide, divide them into three groups of tissue sections with a size of 0.5 cm*0.5 cm at each agarose concentration. During the slicing, slice the tissue into pieces with a large area first, and then trim them to a size of 0.5 cm*0.5 cm. In this way, the tissue sections contaminated with OCT can be removed and the influence of OCT freezing medium on the detection data can be avoided. After the operation is complete, store the cut tissue sections in the refrigerator at −20° C.
(2) Staining of D-2-HG standard substance sections
i. The staining solution is divided into four groups as below:
\textcircled{1} 489.5 μl 1/15M PBS+1 μl 5 mM resazurin+1 μl 1.3 mg/ml FAD-dependent D-2-hydroxyglutarate dehydrogenase
\textcircled{2} 498.5 μl 1/15M PBS+1 μl 5 mM resazurin+1 μl 13 mg/ml FAD-dependent D-2-hydroxyglutarate dehydrogenase
\textcircled{3} 498.33.5 μl 1/15M PBS+1 μl 5 mM resazurin+1 μl 19.5 mg/ml FAD-dependent D-2-hydroxyglutarate dehydrogenase
\textcircled{4} 498.5.5 μl 1/1SM PBS+1 μl 5 mM resazurin+1 μl 26 mg/ml FAD-dependent D-2-hydroxyglutarate dehydrogenase
ii. Add 50 μl of staining solution onto the tissue at the room temperature of 25° C. in the dark environment to cover the D-2-HG standard substance completely, and use the Fluorescence detector to detect the fluorescence of the staining solution on the glass slide every 2s for a total of 300s.
3. Result handling

Draw the scatter diagram by taking the D-2-HG concentration of 0 μmol/g, 0.1 μmol/g, 0.25 μmol/g, 0.5 μmol/g, 1 μmol/g, 1.5 μmol/g, 2.0 μmol/g of D-2-HG by adding 50 μl of the above-mentioned four groups of different staining solution in the standard substance sections with the section thickness of 20 μm as Value x, and the fluorescence value detected by the above method at the time point of 180s as Value y, and form the fitting linear relationship of y=ax+b according to the scatter diagram. There is no significant difference in the standard curves of the four groups of different staining solution (p>0.05). The results indicate that the concentration range of FAD-dependent D-2-hydroxyglutarate dehydrogenase in the kit is 1.3 mg/ml~26 mg/ml.
VIII. Test for the Range of D-2-HG in the Standard substance Section and Detection Time Node in the Standard Curve
1. Fluorescence detector

The fluorescence detector adopts the instrument (BLP-FD-100CS) customized by wave-particle photon, which integrates excitation light, beam collimation, uniform light, extinction, fluorescence collection, fluorescence detection, filtering and signal shielding protection into one unit, with the excitation wavelength of 540 nm, the emission wavelength of 590 nm, the optical density of OD5, the spot diameter of 6.5 mm, the input voltage of 5V, the analog signal output of 0-3.3V, and digital signal output.
2. D-2-HG standard substance treatment:
(1) Production of D-2-HG standard substance: prepare 3% agarose solution containing D-2-HG 0 μmol/g, 0.1 μmol/g, 0.25 μmol/g, 0.5 μmol/g, 1 μmol/g, 1.5 μmol/g, 2.0 μmol/g, 3.0 μmol/g, 4.0 μmol/g and 5.0 μmol/g (20 ml for each) (in order to convert g/cm³, the density of brain tumor tissue refers to 1.054 g/cm³ of normal brain tissue). Use the microwave oven for heating, and heat 3% agarose solution containing D-2-HG at each concentration at the medium baking temperature till it is clear and transparent, then pour it into the gel plate to solidify and take out after half an hour, and cut the solidified agarose into cuboids with a cross-sectional area of 1.5 cm*1.5 cm. Place them in the tissue tray of the freezing microtome (Leica), and cover the agarose gel block with 400 μl of OCT freezing medium. After the OCT freezing medium is frozen as solid in the freezing microtome, cut the agarose gel to reach a thickness of 20 μm, place them on an anti-slip glass slide, divide them into three groups of tissue sections with a size of 0.5 cm*0.5 cm at each agarose concentration. During the slicing, slice the tissue into pieces with a large area first, and then trim them to a size of 0.5 cm*0.5 cm. In this way, the tissue sections contaminated with OCT can be removed and the influence of OCT freezing medium on the detection data can be avoided. After the operation is complete, store the cut tissue sections in the refrigerator at −20° C.
(2) Staining of D-2-HG standard substance sections
i. Preparation of staining solution: 500 μl 1/15M PBS+1 μl 5 mM resazurin+1 μl 13 mg/ml FAD-dependent D-2-hydroxyglutarate dehydrogenase;
ii. Add 50 μl of staining solution onto the tissue at the room temperature of 25° C. in the dark environment to cover the D-2-HG standard substance completely, and use the Fluorescence detector to detect the fluorescence of the staining solution on the glass slide every 2s for a total of 300s.
3. Result handling

Draw the scatter diagram by taking the D-2-HG concentration of 0 μmol/g, 0.1 μmol/g, 0.25 μmol/g, 0.5 μmol/g, 1 μmol/g, 1.5 μmol/g, 2.0 μmol/g, 2.5 μmol/g 3.0 μmol/g, 4.0 μmol/g and 5.0 μmol/g of D-2-HG by adding 50 μl of the staining solution in the standard substance sections with the section thickness of 20 μm as Value x, and the fluorescence value detected by the above method at the time point of 60s and 120s as Value y, and form the fitting linear relationship of y=ax+b according to the scatter diagram. The results are shown in FIG. 7. As the corresponding fluorescence value increases with the increase of reaction time after the section in the standard curve is added with staining solution, the problem of reaching the upper limit of fluorescence detection by the machine will occur. Therefore, we need to choose the appropriate time node range to develop the standard curve. Therefore, when the value detected at the time point of 60s is used for fitting the standard curve, the detectable standard curve range can reach 0 μmol/g~5.0 μmol/g. When the value detected at the time point of 120s is used for fitting the standard curve, the detectable standard curve range can reach 0 μmol/g~3.0 μmol/g. When the value detected at the time point of 180s is used for fitting the standard curve, the detectable standard curve range can reach 0 μmol/g~2.0 μmol/g. However, it can be seen from FIG. 7 that the standard curve fitting R² at 60s is smaller than that at 120s and the standard curve fitting R² at 120s is smaller than that at 180s. Therefore, the detectable standard curve range at 180s is small, but the standard curve accuracy is higher. During the use of the kit, the user can choose the numerical time node of the standard curve according to the own needs.

The present invention is further described in combination with the specific embodiments.

Take eight patient-derived tumor tissues (the acquisition thereof has been approved by the unit ethical organization), number them as #1, #2, #3, #4, . . . , #7, #8, wherein each numbered sample is evenly divided into two parts, one part is detected by using the method in Embodiment 1, and the other part is detected by using the mass spectrometry method of proportional medium.

### Embodiment 1

\textcircled{1} Preparing standard substance sections of D-2-hydroxyglutarate: preparing D-2-hydroxyglutarate into a series of agarose water solution with a concentration of 0 μmol/g, 0.1 μmol/g, 0.25 μmol/g, 0.5 mol/g, 1 μmol/g, 1.5 μmol/g and 2.0 μmol/g (20 ml for each), heating the agarose water solution till reaching the clear and transparent state, pouring the solution onto a gel plate to solidify and take out, cutting it into cuboids with a cross-sectional area of 1.0 cm×1.0 cm, and placing them in a tissue tray of a freezing microtome, covering an agarose gel block with 400 μl of OCT freezing medium; after the OCT freezing medium is frozen as solid in the freezing microtome, cutting the agarose gel to reach a thickness of 20 um, placing them on an anti-slip glass slide, cutting them into cuboids with a cross-sectional area of 0.5 cm×0.5 cm, and storing them in a refrigerator at −20° C. to obtain a series of standard substance sections of D-2-hydroxyglutarate with different concentrations for standby;
three groups of parallel experiments are set for each concentration of standard substance sections;
wherein the concentration of agarose in the aqueous solution is 3%, and the density of brain tumor tissue refers to 1.054 g/cm³ of normal brain tissue).
\textcircled{2} slicing of tissue samples to be detected
taking out the tissue to be detected from the refrigerator at −80° C., placing it onto the ice, and then placing it in the tissue tray of the freezing microtome, adding 400 μl of the OCT freezing medium to the tissue to be detected; after the OCT freezing medium turns white, slicing the tissue to be detected to a thickness of 20 μm, placing it on the anti-slip glass slide, and cutting it into cuboids with a cross-sectional area of 0.5 cm×0.5 cm, so as to obtain the tissue sections to be detected for standby;
Five groups of parallel experiments are set for each tissue sample section to be detected;
\textcircled{3} preparation of staining solution: adding 1 μl of resazurin and 1 μl of FAD-dependent D-2-hydroxyglutarate dehydrogenase to 500 μl of the PBS buffer solution with the mass concentration of 1/15 mol/L to prepare the staining solution;
\textcircled{4} staining of tissue sections

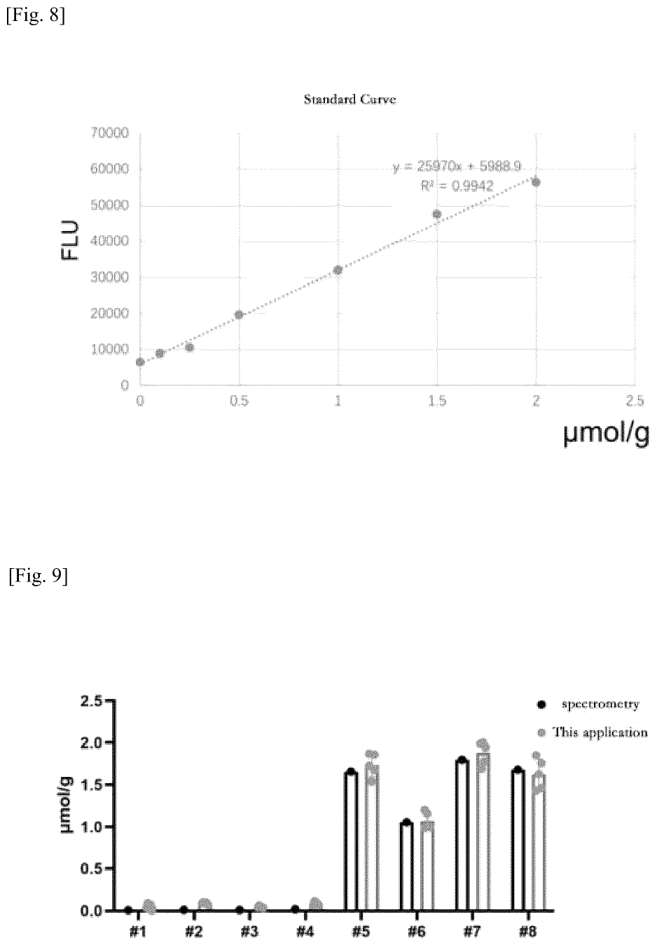

taking 50 μl of the staining solution obtained from Step \textcircled{3} at the room temperature of 25° C. in the dark environment, covering it completely on the tissue sections to be detected and obtained from Step \textcircled{2}, detecting the fluorescence value of the staining solution on the glass slide with a fluorescence detector every 2s, for a total of 300s;
\textcircled{5} data test and result processing of standard substance
placing the standard substance sections of the series of D-2-hydroxyglutarate with different concentrations in Step \textcircled{1} on the glass slide, take 50 μl of the staining solution obtained in Step \textcircled{3} to cover completely at the room temperature of 25° C. at the dark environment, drawing the scatter diagram by taking the average fluorescence value of each concentration detected by the fluorescence detector at 180s as Value y, and D-2-hydroxyglutarate concentration as Value x, and forming the standard substance curve y=ax+b according to the scatter diagram; the results are shown in FIG. 8;
and calculating the content of D-2-hydroxyglutarate in the tissue sections to be detected according to the fluorescence value obtained in Step \textcircled{3}.

### Relative Proportion

Detect the content of D-2-HG in the pathological tissues by means of mass spectrometry.

The test process is as follows:
1. Sample preparation:
(1) Prepare 80% methanol solution and pre-cool it in the refrigerator at 4° C. in advance;
(2) take 100 mg of tissue (8 copies from the same tissue with the embodiments), place it in 1 ml of 80% methanol solution, placing two steel balls in each sample, and crush it with the tissue crusher at 20 times/second, and homogenize for 30 min;
(3) centrifuge at 14,000 rpm and at 4° C. for 30 min, filter the supernatant through a 100 μm sieve, and obtain the supernatant in a unified volume of 750 μl; and
(4) dry the sample completely at (60° C.) in a vacuum concentrator for 2.5 h and then re-suspend with 300 μl of pure water; centrifuge for 13000g*5 min, take the supernatant, place it to the new tube, and store the supernatant in the refrigerator at −80° C.
2. LC-MS/MS mass spectrometry detection
(1) Standard curve (D-2-HG internal standard method): prepare DL-2-hydroxyglutarate disodium salt (13C5, 99%) (purchased from Cambridge Isotope Laboratory, USA) was to be a standard substance with the concentration of 100 nM, 300 nM, 700 nM, 1 μM, 5M, 10 μM, 50M, 100 μM.
(2) The instruments used refer to high performance liquid chromatography LC-20A (Shimadzu, Japan) and Nano high-resolution combined LC-MS instrument (Thermo Fisher, USA). The mobile phase is characterized by adjusting PH to 4.5 with methanol, 0.1% of triethylamine and 5% of acetic acid. The running time is set to 15 min, 20 μl samples are added to the Chirobiotic R column (250*4.6 mm), and the flow rate is set to 0.5 ml/min. On-line detection is performed for the sample substance and sample. The monitoring of D-2-HG mass to charge ratio is converted to 147.0m/z->129.0m/z and the internal standard of D-2-HG is 150.0m/z->132.0m/z.

The detection results are shown in Table 1, and the comparison diagram is drawn as shown in FIG. 9. It can be seen from the results in Table 1 and FIG. 9, the results obtained by this experimental method are consistent with those obtained by mass spectrometry for detection of D-2-hydroxyglutarate, indicating that the accuracy of this experimental method is relatively high. The mass spectrometry method takes 5~6 hours from sample treatment to calculation of the D-2-HG concentration result. The D-2-HG standard substance for the experimental method in this application can be prepared in advance and stored in the refrigerator at −20° C., and it takes up to 15 minutes from sample treatment to calculation of the D-2-HG concentration result, which greatly reduces the time.

Or for the kit obtained by using the method for determination of D-2-hydroxyglutarate concentration in this application, it just needs to take the PBS buffer solution, resazurin solution, FAD-dependent D-2-hydroxyglutarate dehydrogenase solution (the concentration range of the resazurin solution is 0.2 mM~25 mM, and the concentration range of the FAD-dependent D-2-hydroxyglutarate dehydrogenase solution is 1.3 mg/ml~26 mg/ml) as the staining reagent to stain the agarose gel glass slide with the serial concentration of D-2-hydroxyglutarate, and then detect the fluorescence value on the glass slide on the fluorescence detector to make the standard curve, next, stain the sample with the staining reagent, detect the fluorescence value on the glass slide on the fluorescence detector, so as to detect the concentration of D-2-hydroxyglutarate in the sample. For this kit, the time for customers to make the staining reagent and agarose gel containing D-2-hydroxyglutarat glass slide is saved, and only the thickness and cross-sectional area of agarose gel containing D-2-hydroxyglutarat are cut according to the thickness and cross-sectional area of the sample, so as to ensure that the thickness and cross-sectional area of the sample and agarose gel containing D-2-hydroxyglutarat are the same.

TABLE 1
Test Results

| | Content of D-2-HG in each | Average content of D-2-HG in | Mass spectrometry |
|---|---|---|---|
| | tissue section in | each tissue section in | data |
| | Embodiment 1 (μmol/g) | Embodiment 1 (μmol/g) | (μmol/g) |
| #1 | 0.039142 | 0.0502822 | 0.0083 |
| | 0.001602 | | |
| | 0.071418 | | |
| | 0.090493 | | |
| | 0.048756 | | |
| #2 | 0.092553 | 0.0916526 | 0.011556 |
| | 0.100107 | | |
| | 0.094995 | | |
| | 0.072867 | | |
| | 0.097741 | | |
| #3 | 0.044102 | 0.045857 | 0.010272 |
| | 0.062872 | | |
| | 0.03609 | | |
| | 0.044255 | | |
| | 0.041966 | | |
| #4 | 0.11407 | 0.0849076 | 0.017832 |
| | 0.068442 | | |
| | 0.088204 | | |
| | 0.067755 | | |
| | 0.086067 | | |
| #5 | 1.872806 | 1.7356934 | 1.6592 |
| | 1.538547 | | |
| | 1.86098 | | |
| | 1.729116 | | |
| | 1.677018 | | |
| #6 | 1.006244 | 1.0689094 | 1.0552 |
| | 1.154647 | | |
| | 0.986037 | | |
| | 1.200137 | | |
| | 0.997482 | | |
| #7 | 1.945521 | 1.8827406 | 1.798 |
| | 1.778269 | | |
| | 1.694491 | | |
| | 1.990005 | | |
| | 2.005417 | | |
| #8 | 1.75834 | 1.625427 | 1.682 |
| | 1.628109 | | |
| | 1.459805 | | |
| | 1.852236 | | |
| | 1.428643 | | |

1. A method for determination of D-2-hydroxyglutarate concentration, wherein it is characterized by taking FAD-dependent D-2-hydroxyglutarate dehydrogenase, resazurin and buffer solution system as the working solution to realize the detection for D-2-hydroxyglutarate in the solid tissue sections;
the specific steps are as follows:
\textcircled{1} preparing standard substance sections of D-2-hydroxyglutarate: adding the D-2-hydroxyglutarate into agarose solution, heating and stirring till the solution became clear, pouring the solution onto a gel plate to solidify and take out, cutting it into cuboids with a cross-sectional area of 0.1~1.5 cm×0.1~1.5 cm, and placing them in a tissue tray of a freezing microtome, and covering the agarose gel block with an OCT freezing medium; after the OCT freezing medium is frozen as solid in the freezing microtome, cutting the agarose gel to reach a thickness of 5~25 μm, placing them on an anti-slip glass slide, cutting them into cuboids with a cross-sectional area of 0.1~1 cm×0.1~1 cm, and storing them in a refrigerator at −20° C. to obtain a series of standard substance sections of D-2-hydroxyglutarate with different concentrations for standby;
wherein the ratio of D-2-hydroxyglutarate to agarose solution is 0~5.0 μmol: 1 g;
three groups of parallel experiments are set for each concentration of standard substance sections;
\textcircled{2} slicing of tissue samples to be detected
taking out the tissue to be detected from the refrigerator at −80° C., placing it onto the ice, and then placing it in the tissue tray of the freezing microtome, adding the OCT freezing medium to the tissue to be detected; after the OCT freezing medium turns white, slicing the tissue to be detected to a thickness of 5~25 μm, placing it on the slides, and cutting it into a square with a cross-sectional area of 0.1~1 cm×0.1~1 cm, which have the same cross-sectional area as the standard substance section, so as to obtain the tissue sections to be detected for standby;
three groups of parallel experiments are set for each tissue sample section to be detected;
\textcircled{3} preparation of staining solution: the staining solution is a buffer solution with the pH of 7.0~8.0, containing resazurin and FAD-dependent D-2-hydroxyglutarate dehydrogenase;
the buffer solution is Tris-Hcl buffer solution, PBS buffer solution or HEPES buffer solution, and the concentration range of the resazurin is 0.5 μM~200 μM, and the concentration range of the FAD-dependent D-2-hydroxyglutarate dehydrogenase is 1.3 ug/mL~520 μg/ml;
\textcircled{4} staining of tissue sections
taking 15 μl~100 μl of the staining solution obtained from Step \textcircled{3}, covering it completely on the tissue sections to be detected and obtained from Step \textcircled{2}, detecting the fluorescence value of the staining solution on the glass slide with a fluorescence detector for 1~10 minutes;
\textcircled{5} data test and result processing of standard substance
placing the standard substance sections of a series of D-2-hydroxyglutarate with different concentrations in Step \textcircled{1} on the glass slide, taking 15 μl~100 μl of the staining solution obtained in Step \textcircled{3} for covering completely, detecting with the fluorescence detector immediately, wherein the fluorescence detector is characterized by the excitation wavelength of 540 nm and the emission wavelength of 590 nm; taking the value when the fluorescence detector detects for 1~10 minutes as the fluorescence value of the staining solution on the slides; drawing the scatter diagram by taking the average fluorescence value of each concentration detected by the fluorescence detector as Value y, and the concentration of D-2-hydroxyglutarate as Value x, and forming the standard substance curve y=ax+b according to the scatter diagram;
and calculating the content of D-2-hydroxyglutarate in the tissue sections to be detected according to the fluorescence value obtained in Step \textcircled{4}.

2. The method for determination of D-2-hydroxyglutarate concentration according to claim 1, wherein the concentration of D-2-hydroxyglutarate in the standard substance section is a gradient concentration in the range of 0~5.0 μmol/g.

3. The method for determination of D-2-hydroxyglutarate concentration according to claim 1, wherein the staining solution is a PBS buffer solution with a pH of 7.4, containing resazurin with a concentration of 10 AM and 26 μg/ml of FAD-dependent D-2-hydroxyglutarate dehydrogenase.

4. The method for determination of D-2-hydroxyglutarate concentration according to claim 1, wherein after the staining solution is added to the standard substance section and the tissue section to be detected, the detection time of the fluorescence detector is the same, 1~3 minutes.

5. A kit obtained by using the method for determination of D-2-hydroxyglutarate concentration according to claim 1, containing buffer solution with pH7.0~8.0, mother liquor of resazurin, mother liquor of FAD-dependent D-2-hydroxyglutarate dehydrogenase, and agarose gel slides containing the serial concentration of D-2-hydroxyglutarate; the concentration range of the mother liquor in the kit is 0.2 mM~25 mM, the concentration range of the mother liquor of FAD-dependent D-2-hydroxyglutarate dehydrogenase in the kit is 1.3 mg/ml~26 mg/ml; and the buffer solution is Tris-Hcl buffer solution, PBS buffer solution or HEPES buffer solution.

6. The kit obtained by using the method for determination of D-2-hydroxyglutarate concentration according to claim 5, wherein the agarose gel containing the serial concentration of D-2-hydroxyglutarate is placed on the slides, and the concentration range of D-2-hydroxyglutarate in the agarose gel is 0~5.0 μmol/g.

7. The kit obtained by using the method for determination of D-2-hydroxyglutarate concentration according to claim 6, wherein the concentrations of D-2-hydroxyglutarate in the agarose gel are 0 μmol/g, 0.1 μmol/g, 0.25 μmol/g, 0.5 μmol/g, 1.0 μmol/g, 1.5 μmol/g and 2.0 μmol/g.

8. The kit obtained by using the method for determination of D-2-hydroxyglutarate concentration according to claim 5, wherein the agarose gel is rectangle, with the thickness of 5~25 μm and the cross-sectional area of 0.1~1 cm×0.1~1 cm, and the size and thickness of the agarose gel are adjusted according to the size of the tissue to be detected.
